(19) United States
(12) Patent Application Publication
(10) Pub. No.: US 2013/0116196 A1
(43) Pub. Date: May 9, 2013

(54) MUSCLE FATIGUE SUBSTANCE CYTOKINES AND METHODS OF INHIBITING TUMOR GROWTH THEREWITH

(71) Applicant: Translational Genomics Research Institute, Phoenix, AZ (US)

(72) Inventors: Ruben M. Munoz, Phoenix, AZ (US); Haiyon Han, Chandler, AZ (US); Daniel D. von Hoff, Scottsdale, AZ (US); Stanley Hoffman, Phoenix, AZ (US)

(73) Assignee: TRANSLATIONAL GENOMICS RESEARCH INSTITUTE, Phoenix, AZ (US)

(21) Appl. No.: 13/669,393

(22) Filed: Nov. 5, 2012

Related U.S. Application Data
(60) Provisional application No. 61/555,433, filed on Nov. 3, 2011.

(51) Int. Cl. A61K 38/19; C12N 5/09; A61P 35/00

(52) U.S. Cl. 514/19.3; 435/375

(57) ABSTRACT

Compositions and methods for inhibiting tumor growth and tumor cell proliferation are provided. In general, the cytokines useful with the present invention are selected from the group consisting of LIX (or CXCL5), TIMP-1, and sICAM.

### CROSS REFERENCE

This application is related to and claims the priority benefit of U.S. Provisional application 61/555,433, filed on Nov. 3, 2011, the teachings and content of which are incorporated by reference herein.

### FIELD OF THE INVENTION

The present invention is concerned with substances and compositions that inhibit tumor growth. More particularly, the present invention is concerned with cytokines released during muscle activity wherein such cytokines inhibit tumor growth. Still more particularly, the present invention is concerned with methods for obtaining and using such cytokines. Even more particularly, the present invention is concerned with compositions that include one or more of LIX (also known as CXCL5), TIMP-1, and sICAM and methods of using one or more of these cytokines to inhibit tumor growth.

### BACKGROUND OF THE INVENTION

It has been scientifically established by Nelson and others that regular physical exercise can reduce cancer risk by as much as 40% (Newton R U, Galvao D A: Exercise in prevention and management of cancer; Curr Treat Options Oncol 9:135-46, 2008, the teachings and content of which are hereby incorporated by reference). In 1960, Hoffman and colleagues exercised tumor-bearing rats to fatigue, causing tumor reduction. They then electrically stimulated rat rectus femoris muscles to fatigue, causing the release of a substance that inhibited growth of transplanted rat tumors. They called this substance Fatigue Substance or F-Substance (Hoffman S A, Paschkis K E, Debias D A, et al: The influence of exercise on the growth of transplanted rat tumors; Cancer Res 22:597-9, 1962, the teachings and content of which are hereby incorporated by reference). Studies continued until 1978, determining that the substance inhibited tumor growth without involving surrounding tissue or reducing carcass weight and that different concentrations of the substance were effective against various tumors. It was also found to be heat sensitive and could be frozen for one year without losing its effectiveness and was probably a polypeptide with a molecular weight of 40,000 to 60,000 (Hoffman S A, DeBias D A, Cantarow A B, et al: Effect of fatigue substance on transplanted rat tumors; Surg Forum 19:85-7, 1968, the teachings and content of which are hereby incorporated by reference).

### SUMMARY OF THE INVENTION

The present invention solves the problems inherent in the prior art and provides a distinct advance in the state of the art by providing cytokines useful for inhibiting tumor growth.

In one aspect of the present invention, a composition for reducing tumor growth is provided. In preferred forms, the composition comprises an isolated cytokine, and especially an isolated cytokine selected from the group consisting of LIX (or CXCL5), TIMP-1, sICAM, and combinations thereof. In other preferred forms, the composition includes at least two or all three of the isolated cytokines selected from the group consisting of LIX (or CXCL5), TIMP-1, and sICAM. The cytokines used in the present invention may be lyophilized prior to use. When the cytokines are lyophilized, they are preferably reconstituted prior to use. Some preferred reconstitution aqueous solutions or liquids are water, saline, PBS, plasma based solutions, or cell growth media.

In preferred forms, the composition may also include additional components known to those of skill in the art (see also Remington's Pharmaceutical Sciences. (1990). 18th ed. Mack Publ., Easton). Additionally, the composition may include one or more pharmaceutically-acceptable carriers. As used herein, “a pharmaceutically-acceptable carrier” includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

Those of skill in the art will understand that the composition used herein may incorporate known injectable, physiologically acceptable sterile solutions. For preparing a ready-to-use solution for parenteral injection or infusion, aqueous isotonic solutions, e.g., saline or corresponding plasma protein solutions, are readily available.

In another aspect of the present invention, a method of inhibiting tumor growth is provided. In general, the method comprises the step of contacting a tumor with a composition including an isolated cytokine. In preferred forms, the isolated cytokine is selected from the group consisting of LIX (or CXCL5), TIMP-1, sICAM, and combinations thereof. In other preferred forms, the composition includes at least two or all three of the isolated cytokines selected from the group consisting of LIX (or CXCL5), TIMP-1, and sICAM. The cytokines used in the present invention may be lyophilized. When the cytokines are lyophilized, they are preferably reconstituted prior to use. Some preferred reconstitution aqueous solutions or liquids are water, saline, PBS, plasma based solutions, or cell growth media.

In another aspect of the present invention, a method of inhibiting tumor growth is provided. In general, the method comprises the step of administering a composition including an isolated cytokine to a subject in need of tumor growth inhibition. In preferred forms, the isolated cytokine is selected from the group consisting of LIX (or CXCL5), TIMP-1, sICAM, and combinations thereof. In other preferred forms, the composition includes at least two or all three of the isolated cytokines selected from the group consisting of LIX (or CXCL5), TIMP-1, and sICAM. The cytokines used in the present invention may be lyophilized. When the cytokines are lyophilized, they are preferably reconstituted prior to use. Some preferred reconstitution aqueous solutions or liquids are water, saline, PBS, plasma based solutions, or cell growth media. In some preferred forms, the cytokines are combined with a pharmaceutically-acceptable carrier prior to administration. Administration can be in any conventional form and can be locally administered or systemically administered. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months and in different dosages. Preferably the subject is an animal, more preferably the subject is a mammal, and more preferably the subject is a human.

In another aspect of the present invention, a method of inhibiting tumor cell proliferation is provided. In general, the method comprises the step of contacting a tumor cell with a composition including an isolated cytokine. In preferred forms, the isolated cytokine is selected from the group consisting of LIX (or CXCL5), TIMP-1, sICAM, and combinations thereof. In other preferred forms, the composition includes at least two or all three of the isolated cytokines selected from the group consisting of LIX (or CXCL5), TIMP-1, and sICAM. The cytokines used in the present invention may be lyophilized. When the cytokines are lyophilized, they are preferably reconstituted prior to use. Some preferred reconstitution aqueous solutions or liquids are water, saline, PBS, plasma based solutions, or cell growth media.

In another aspect of the present invention, a method of inhibiting tumor cell proliferation is provided. In general, the method comprises the step of administering a composition including an isolated cytokine to a subject in need of tumor growth inhibition. In preferred forms, the isolated cytokine is selected from the group consisting of LIX (or CXCL5), TIMP-1, sICAM, and combinations thereof. In other preferred forms, the composition includes at least two or all three of the isolated cytokines selected from the group consisting of LIX (or CXCL5), TIMP-1, and sICAM. The cytokines used in the present invention may be lyophilized. When the cytokines are lyophilized, they are preferably reconstituted prior to use. The reconstitution can be in any conventional aqueous solution including, for example, water, saline, PBS, plasma-based solutions, cell growth media, and the like. In some preferred forms, the cytokines are combined with a pharmaceutically-acceptable carrier prior to administration. Administration can be in any conventional form and can be locally administered or systemically administered. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months and in different dosages. Preferably the subject is a mammal and more preferably, the subject is a human.

In another aspect of the present invention, a method for estimating a subject's risk level of developing a cancer is provided. In general, the method includes the steps of determining the amount of at least one cytokine present in a sample derived from a subject and comparing the determined amount with a standard or control with a known risk level. In preferred forms, the cytokines are selected from the group consisting of LIX (or CXCL5), TIMP-1, and sICAM.

### DESCRIPTION OF THE FIGURES

FIG. 1 is a collection of 2 graphs illustrating the effect of F-substance on muscle contraction upon electric stimulation. The contraction ability of a muscle injected with 1 ml of substance extracted from stimulated muscle (A) is greatly diminished compared to that injected with the same volume of saline solution. The hashmarks at the bottom of the graph represent 3.00, 47.84, 95.67, and 143.51, respectively. (B). Y axis: contraction force (grams); X-axis: time (seconds). The hashmarks at the bottom of the graph represent 3.00, 47.03, 94.06, and 141.09, respectively;

FIG. 2 is a collection of 2 graphs illustrating the growth inhibitory effect of the fatigue substances against MCF-7 cells. Graph A shows the concentration dependent effect of F-substance on MCF-7 cell growth as determined by SRB assay. Graph B shows the real time cell growth effect of the F-substance on MCF-7 using impedance measurements;

FIG. 3 is a photograph illustrating the identification of cytokines differentially produced by unstimulated (A) and stimulated (B) muscles. Each cytokine antibody was printed in duplicate. The four spots in the first column and two spots in the last column serve as positive and membrane direct controls. The circles in (A) are for cytokines showing higher intensity in the unstimulated muscle than the stimulated muscle. The circles in (B) are for cytokines showing higher intensity in the stimulated muscle than the unstimulated muscle;

FIG. 4 is a graph illustrating the quantification of the rat cytokines antibody array of FIG. 3. The single intensity of each spot was quantified by densitometry. The relative intensity (Y-Axis) was calculated by dividing the average intensities of the positive controls from the spot intensities of the individual cytokines. For each cytokine, the relative intensity for unstimulated muscle is on the left and the relative intensity for the stimulated muscle is on the right. The cytokines that are the most significantly higher in the stimulated muscle are LIX, TIMP-1, and sICAM-1; and

FIG. 5 is a photograph of a Western Blot for validation of TIMP-1 levels. In FIG. 5, B lanes are for biotinylated protein markers; S lanes are for stimulated extract; U lanes are for unstimulated extract; and P lanes are for prestained protein markers.

### DETAILED DESCRIPTION OF THE INVENTION

The invention identifies the composition and activity of F-Substance. As noted herein, it was determined that F-Substance contains cytokines, three of which were at significantly higher levels in the substance isolated from the stimulated muscle than in the substance isolated from the unstimulated muscle.

F-Substance has shown to exert anti-tumor activity in both rat and human tumors. Without limiting the current invention to any specific mode of action, it is believed that the fatigue substance induces apoptosis as opposed to necrosis.

Cytokines are known to play important roles in a number of biological processes including innate immunity, apoptosis, angiogenesis, cell growth, and differentiation. Many of these processes are involved in cancer pathogenesis and therapy (Dranoff G: Cytokines in cancer pathogenesis and cancer therapy; Nat Rev Cancer 4:11-22, 2004, the teachings and content of which are hereby incorporated by reference). LIX (Lipopolysaccharide-induced CXC chemokine or CXCL5) is member of the CXC chemokine family which are potent neutrophil chemoattractants (Chandrasekar B, Melby P C, Sarau H M, et al: Chemokine-cytokine cross-talk; The ELR+CXC chemokine LIX (CXCL5) amplifies a proinflammatory cytokine response via a phosphatidylinositol 3-kinase-NF-kappa B pathway. J Biol Chem 278:4675-86, 2003, the teachings and content of which are hereby incorporated by reference). TMIP-1 (Tissue inhibitor of metalloproteinases-1) is a naturally occurring inhibitor of metalloproteinases (Brew K, Dinakarpandian D, Nagase H: Tissue inhibitors of metalloproteinases: evolution, structure and function; Biochim Biophys Acta 1477:267-83, 2000, the teachings and content of which are hereby incorporated by reference). The TIMPs inhibit tumorigenesis, cellular invasion, metastasis and angiogenesis. However, they have also been known to promote tumor growth and inhibit apoptosis (Stetler-Stevenson W G: Tissue inhibitors of metalloproteinases in cell signaling: metalloproteinase-independent biological activities; Sci Signal 1:re6, 2008, the teachings and content of which are hereby incorporated by reference). sICAM-1 (Soluble intercellular adhesion molecule-1), a counter-receptor for the leukocyte integrin, lymphocyte function-associated antigen (LFA-1), has been reported to inhibit the interaction between T cells and tumors and, block NK cell-mediated toxicity, suggesting a cancer promoting role (Witkowska A M, Borawska M H: Soluble intercellular adhesion molecule-1 (sICAM-1): an overview; Eur Cytokine Netw 15:91-8, 2004, the teachings and content of which are hereby incorporated by reference).

A marker may be any molecular structure produced by a cell, expressed inside the cell, accessible on the cell surface, or secreted or released by the cell. A marker may be any protein, carbohydrate, fat, nucleic acid, catalytic site, or any combination of these, such as, an enzyme, glycoprotein, cell membrane, virus, cell, organ, organelle, or any uni- or multimolecular structure or any other such structure now known or yet to be disclosed whether alone or in combination. A marker may also be called a target and the terms are used interchangeably. A marker may be represented by a protein. The concept of a marker is not limited to the products of the exact nucleic acid sequence or protein sequence by which it may be represented. Rather, a marker encompasses all molecules that may be detected by a method of assessing the expression or release of the marker. In one preferred embodiment of the invention, the marker is a cytokine selected from the group consisting of LIX (also known as CXCL5), TIMP-1, sICAM, and combinations thereof.

“Isolated” means altered by the hand of man from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

Expression or release of a marker such as a cytokine from stimulated muscles may be assessed by any number of methods. Examples of such methods include any detection method including the following nonlimiting examples, flow cytometry, immunohistochemistry, ELISA, Western blot, and immunoaffinity chromatograpy, HPLC, mass spectrometry, protein microarray analysis, PAGE analysis, isoelectric focusing, 2-D gel electrophoresis, or any enzymatic assay or any method that uses a protein reagent, nucleic acid reagent, or other reagent capable of specifically binding to or otherwise recognizing a specific nucleic acid or protein marker.

Other methods used to assess expression or release of a marker such as a cytokine from stimulated muscles include the use of natural or artificial ligands capable of specifically binding a marker. Such ligands include antibodies, antibody complexes, conjugates, natural ligands, small molecules, nanoparticles, or any other molecular entity capable of specific binding to a marker. The term “antibody” is used herein in the broadest sense and refers generally to a molecule that contains at least one antigen binding site that immunospecifically binds to a particular antigen target of interest. Antibody thus includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term thus includes full length antibodies and/or their variants as well as immunologically active fragments thereof, thus encompassing, antibody fragments capable of binding to a biological molecule (such as an antigen or receptor) or portions thereof, including but not limited to Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

Antibodies may be monoclonal, polyclonal, or any antibody fragment including a Fab, F(ab)2, Fv, scFv, phage display antibody, peptibody, multispecific ligand, or any other reagent with specific binding to a marker. Ligands may be associated with a label such as a radioactive isotope or chelate thereof, dye (fluorescent or nonfluorescent), stain, enzyme, metal, or any other substance capable of aiding a machine or a human eye from differentiating a cell expressing or releasing a marker from a cell not expressing or releasing a marker. Additionally, expression or release of a marker such as a cytokine from stimulated muscles may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

Differential expression encompasses any detectable difference between the expression or release of a marker such as a cytokine from stimulated muscle in one sample relative to the expression or release of a marker such as a cytokine in another sample. Differential expression may be assessed by a detector, an instrument containing a detector, by aided or unaided human eye, or any other method that may detect differential expression. Examples include but are not limited to differential staining of cells in an IHC assay configured to detect a marker, differential detection of bound RNA on a microarray to which a sequence capable of binding to the marker is bound, differential results in measuring RT-PCR measured in ACt or alternatively in the number of PCR cycles necessary to reach a particular optical density at a wavelength at which a double stranded DNA binding dye (e.g., SYBR Green) incorporates, differential results in measuring label from a reporter probe used in an RT-PCR reaction, differential detection of fluorescence labels on cells using a flow cytometer, differential intensities of bands in a Northern blot, differential intensities of bands in an RNAse protection assay, differential cell death measured by apoptotic markers, differential cell death measured by shrinkage of a tumor, or any method that allows a detection of a difference in signal between one sample or set of samples and another sample or set of samples.

A label used to facilitate the differential expression detection may be any substance capable of aiding a machine, detector, sensor, device, or enhanced or unenhanced human eye from differentiating a labeled composition from an unlabeled composition. Examples of labels include but are not limited to: a radioactive isotope or chelate thereof, dye (fluorescent or nonfluorescent), stain, enzyme, or nonradioactive metal. Specific examples include but are not limited to: fluorescein, biotin, digoxigenin, alkaline phosphatese, biotin, streptavidin, 3H, 14C, 32P, 35S, or any other compound capable of emitting radiation, rhodamine, 4-(4′-dimethylamino-phenylazo)benzoic acid (“Dabcyl”), 4-(4′-dimethylamino-phenylazo)sulfonic acid (sulfonyl chloride) (“Dabsyl”), 5-((2-aminoethyl)-amino)-naphtalene-1-sulfonic acid (“EDANS”), Psoralene derivatives, haptens, cyanines, acridines, fluorescent rhodol derivatives, cholesterol derivatives, ethylenediaminetetraaceticacid (“EDTA”) and derivatives thereof, or any other compound that may be differentially detected. The label may also include one or more fluorescent dyes optimized for use in genotyping. Examples of such dyes include but are not limited to: dR110, 5-FAM, 6FAM, dR6G, JOE, HEX, VIC, TET, dTAMRA, TAMRA, NED, dROX, PET, BHQ+, Gold 540, MGB and LIZ.

The expression or release of the marker such as a cytokine in a sample may be compared to a level of expression predetermined to predict the presence or absence of a particular cellular or physiological characteristic. The level of expression or release may be derived from a single control or a set of controls. A control may be any sample with a previously determined level of expression or release. A control may comprise material within the sample or material from sources other than the sample. Alternatively, the expression or release of a marker in a sample may be compared to a control that has a level of expression or release predetermined to signal or not signal a cellular or physiological characteristic. This level of expression or release may be derived from a single source of material including the sample itself or from a set of sources. Comparison of the expression or release of the marker in the sample to a particular level of expression or release results in a prediction that the sample exhibits or does not exhibit the cellular or physiological characteristic.

Prediction of a cellular or physiological characteristic includes the prediction of any cellular or physiological state that may be predicted by assessing the expression or release of a marker. Examples include the likelihood that one or more diseases is present or absent, the likelihood that a present disease will progress, remain unchanged, or regress, the likelihood that a disease will respond or not respond to a particular therapy, or any other disease outcome. Further examples include the likelihood that a cell will move, senesce, apoptose, differentiate, metastasize, or change from any state to any other state or maintain its current state.

Expression or release of a marker in a sample may be more or less than that of a level predetermined to predict the presence or absence of a cellular or physiological characteristic. The expression or release of the marker in the sample may be more than 1,000,000×, more than 100,000×, more than 10,000×, more than 1000×, more than 100×, more than 10×, more than 5×, more than 2×, more than 1×, less than 1×, less than 0.5×, less than 0.1× less than 0.01×, less than 0.001×, less than 0.0001×, less than 0.00001×, less than 0.000001×, less than 0.0000001× or any value more or less than that of a level predetermined to predict the presence or absence of a cellular or physiological characteristic.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

Determining the level of expression or release that signifies a physiological or cellular characteristic may be assessed by any of a number of methods. The skilled artisan understands that numerous methods may be used to select a level of expression or release for a particular marker or a plurality of markers that signifies a particular physiological or cellular characteristic. In diagnosing the presence of a disease, a threshold value may be obtained by performing the assay method on samples obtained from a population of patients having a certain type of disease (cancer for example), and from a second population of subjects that do not have the disease. In assessing disease outcome or the effect of treatment, a population of patients, all of which have a disease such as cancer, may be followed for a period of time. After the period of time expires, the population may be divided into two or more groups. For example, the population may be divided into a first group of patients whose disease progresses to a particular endpoint and a second group of patients whose disease does not progress to the particular endpoint. Examples of endpoints include disease recurrence, death, metastasis or other states to which disease may progress. If expression or release of the marker in a sample is more similar to the predetermined expression or release of the marker in one group relative to the other group, the sample may be assigned a risk of having the same outcome as the patient group to which it is more similar.

In addition, one or more levels of expression or release of the marker may be selected that provide an acceptable ability of its ability to signify a particular physiological or cellular characteristic. Examples of such characteristics include identifying or diagnosing a particular disease, assessing a risk of outcome or a prognostic risk, or assessing the risk that a particular treatment will or will not be effective.

For any particular marker, a distribution of marker expression or release levels for subjects with and without a disease may overlap. The area of overlap indicates where the test cannot distinguish the two groups. This indicates that the test does not absolutely distinguish between the two populations with complete accuracy. Therefore, Receiver Operating Characteristic curves, or “ROC” curves, may be calculated by plotting the value of a variable versus its relative frequency in two populations. An ROC curve, is a graphical plot of the sensitivity, or true positive rate (TPR), vs. false positive rate (FPR), for a binary classifier system as its discrimination threshold is varied. The ROC can also be represented equivalently by plotting the fraction of true positives out of the positives (TPR) vs. the fraction of false positives out of the negatives (FPR). TPR determines a classifier or a diagnostic test performance on classifying positive instances correctly among all positive samples available during the test. FPR, on the other hand, defines how many incorrect positive results occur among all negative samples available during the test.

A ROC space is defined by FPR and TPR as x and y axes respectively, which depicts relative trade-offs between true positive (benefits) and false positive (costs). Since TPR is equivalent with sensitivity and FPR is equal to 1—specificity, the ROC graph is sometimes called the sensitivity vs (1—specificity) plot. Each prediction result or one instance of a confusion matrix represents one point in the ROC space. The diagonal divides the ROC space. Points above the diagonal represent good classification results, points below the line poor results. The best possible prediction method would yield a point in the upper left corner or coordinate (0,1) of the ROC space, representing 100% sensitivity (no false negatives) and 100% specificity (no false positives). The (0,1) point is also called a perfect classification.

When a threshold for classification in a binary classifier system is selected, expression or release of the marker in the sample above the threshold indicates the sample is similar to one group and expression or release of the marker below the threshold indicates the sample is similar to the other group. The area under the ROC curve (AUC, “Area Under Curve”) is a measure of the probability that the expression or release correctly indicated the similarity of the sample to the proper group. ROC analysis provides tools to select possibly optimal models and to discard suboptimal ones independently from (and prior to specifying) the cost context or the class distribution (Hanley et al., Radiology 143: 29-36 (1982)).

Additionally, levels of expression or release may be established by assessing the expression or release of a marker in a sample from one patient, assessing the expression or release of additional samples from the same patient obtained later in time, and comparing the expression or release of the marker from the later samples with the initial sample or samples. This method may be used in the case of markers that indicate, for example, progression or worsening of disease or lack of efficacy of a treatment regimen or remission of a disease or efficacy of a treatment regimen.

Other methods may be used to assess how accurately the expression or release of a marker signifies a particular physiological or cellular characteristic. Such methods include a positive likelihood ratio, negative likelihood ratio, odds ratio, and/or hazard ratio. In the case of a likelihood ratio, the likelihood that the expression or release of the marker would be found in a sample with a particular cellular or physiological characteristic is compared with the likelihood that the expression or release of the marker would be found in a sample lacking the particular cellular or physiological characteristic.

An odds ratio measures effect size and describes the amount of association or non-independence between two groups. An odds ratio is the ratio of the odds of a marker being expressed or released in one set of samples versus the odds of the marker being expressed or released in the other set of samples. An odds ratio of 1 indicates that the event or condition is equally likely to occur in both groups. An odds ratio grater or less than 1 indicates that expression or release of the marker is more likely to occur in one group or the other depending on how the odds ratio calculation was set up.

A hazard ratio may be calculated by an estimate of relative risk. Relative risk is the chance that a particular event will take place. It is a ratio of the probability that an event such as development or progression of a disease will occur in samples that exceed a threshold level of expression or release of a marker over the probability that the event will occur in samples that do not exceed a threshold level of expression or release of a marker. Alternatively, a hazard ratio may be calculated by the limit of the number of events per unit time divided by the number at risk as the time interval decreases. In the case of a hazard ratio, a value of 1 indicates that the relative risk is equal in both the first and second groups; a value greater or less than 1 indicates that the risk is greater in one group or another, depending on the inputs into the calculation.

Additionally, multiple threshold levels of expression or release may be determined. This can be the case in so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk of a disease developing or signifying a physiological or cellular state. A risk may be assigned based on which “bin” a test subject falls into.

The invention contemplates assessing the expression or release of the marker in any biological sample from which the expression or release may be assessed. The type of biological sample, the subject comprising the sample, and the manner in which the sample is collected can and will vary, and is known to the skilled in the art.

The term “sample” or “biological sample” is used in its broadest sense. Depending upon the embodiment of the invention, for example, a sample may comprise a bodily fluid including whole blood, serum, plasma, urine, saliva, cerebral spinal fluid, semen, vaginal fluid, pulmonary fluid, tears, perspiration, mucus and the like; an extract from a cell, chromosome, organelle, or membrane isolated from a cell; a cell; genomic DNA, RNA, or cDNA, in solution or bound to a substrate; a tissue; a tissue print or any other material isolated in whole or in part from a living subject. Such samples include, but are not limited to, tissue isolated from primates, e.g., humans, or rodents, e.g., mice, and rats. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish. In preferred forms, the biological sample is blood.

A biological sample for use is obtained in methods described in this invention. Most often, this will be done by removing a sample from a subject, but can also be accomplished by using previously isolated samples (e.g., isolated by another person, at another time, and/or for another purpose), or by performing the methods of the invention in vivo. Archival tissues, having treatment or outcome history, will be particularly useful.

Examples of sources of samples include but are not limited to biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the invention, the sample comprises a fluid sample, such as peripheral blood, lymph fluid, ascites, serous fluid, pleural effusion, sputum, cerebrospinal fluid, amniotic fluid, lacrimal fluid, stool, or urine. Samples include single cells, whole organs or any fraction of a whole organ, in any condition including in vitro, ex vivo, in vivo, post-mortem, fresh, fixed, or frozen.

The term “subject” is used in its broadest sense. In a preferred embodiment, the subject is a mammal. Non-limiting examples of mammals include humans, dogs, cats, horses, cows, sheep, goats, and pigs. Preferably, a subject includes any human or non-human mammal, including for example: a primate, cow, horse, pig, sheep, goat, dog, cat, or rodent, capable of developing cancer including human patients that are suspected of having cancer, that have been diagnosed with cancer, or that have a family history of cancer. Methods of identifying subjects suspected of having cancer include but are not limited to: physical examination, family medical history, subject medical history, endometrial biopsy, or a number of imaging technologies such as ultrasonography, computed tomography, magnetic resonance imaging, magnetic resonance spectroscopy, or positron emission tomography.

Cancer cells include any cells derived from a tumor, neoplasm, cancer, precancer, cell line, malignancy, or any other source of cells that have the potential to expand and grow to an unlimited degree. Cancer cells may be derived from naturally occurring sources or may be artificially created. Cancer cells may also be capable of invasion into other tissues and metastasis. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Examples of cancers that could serve as sources of cancer cells include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may serve as sources of cancer cells include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

### EXAMPLES

The following non-limiting examples are included to illustrate the invention, and they are not intended to limit the scope of the claims.

### Example 1

### Preparation of Substances Released from Stimulated Muscles

To prepare fatigue substances released from stimulated muscles, a system that allows for the electrical stimulation of rat rectus femoris muscles, monitoring muscle contraction, and collecting substances released from the muscle was assembled. The system is a modified version of the one described by Hoffman and colleagues. It consists of a stimulator (Model SD9, Grass Instruments, West Warwick, R.I.), a force transducer (Biopac Systems Inc., Aero Camino Goleta, Calif.), a data acquisition unit (Model MP35, Biopac Systems Inc.), and a water bath.

The right and left rectus femoris muscles were removed from 8-9 week old Wistar rats and rinsed with saline solution to remove any trace of blood on the surface. The muscles were clamped between two electrode clips that carried current from the stimulator. They were then placed in a conical tube containing 30 ml of saline solution, which was placed in a 37° C. water bath. After applying 30 g of force to the muscles by the force condenser, 9 volts of electricity were applied to one of the muscles for 180 sec and the other muscle from the same rat was left in the saline unstimulated for the same period of time as a control. The contraction pattern of the stimulated muscle was recorded by the transducer. The saline solutions were then spun at 2,000 rpm for 5 minutes to remove blood cells. The samples were then dialyzed in 3.5K MWCO dialysis cassettes (Pierce Biotechnology, Rockford, Ill.) against in 5 liters of dH₂O changed four times. Samples were finally lyophilized at −20° C. until dry.

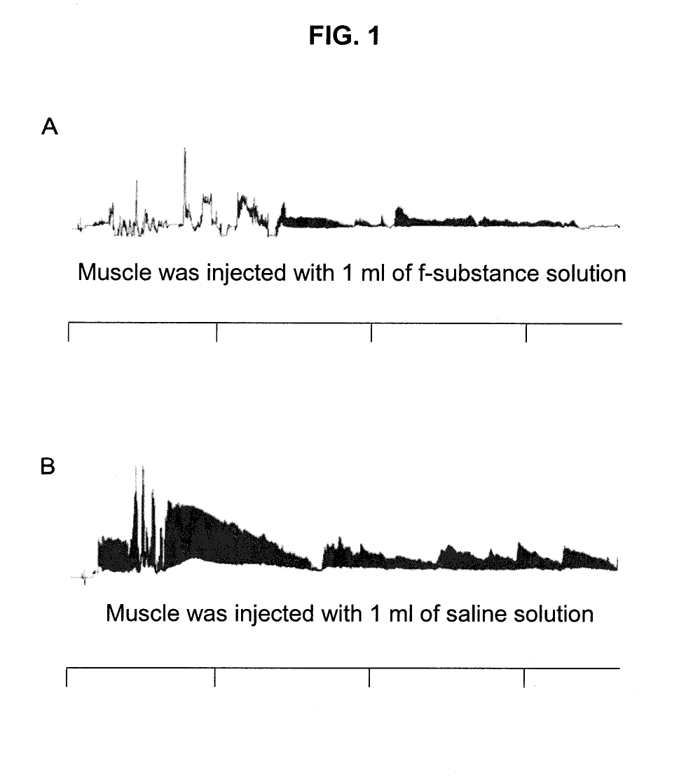

The lyophilized substance was reconstituted in cell culture media (RPMI with 10% FBS) before use. To determine the “fatiguing” effect of the isolated substances, the lyophilized sample was injected into a fresh muscle and the contraction pattern upon electrically stimulation was recorded. As shown in FIG. 1A, the muscle injected with substances collected from stimulated muscles significantly diminished the contracting ability of the muscle (causing a fatigue effect) compared to the muscle injected with the same volume of saline solution (FIG. 1B). This result indicates that the substances that we obtained from the stimulated muscle can cause fresh muscles to appear “fatigued”.

### Example 2

### Evaluation of the Substances Released from the Stimulated Muscles

To evaluate the F-substances released from the stimulated muscles in cancer cell line models, the anti-tumor effect of the substances was measured using the growth of two cancer cell lines, Mia PaCa-2 (a pancreatic cancer cell line) and MCF-7 (a breast cancer cell line). The cancer cells were seeded in 96-well cell culture plates at 2,000 cells/well and treated with different amounts of substances extracted from stimulated or unstimulated rat muscles for 72 hours. At the end of treatment period, cell viability was measured using the sulforhodamine B (SRB) assay (Xie L, Kassner M, Munoz R M, et al: Kinome-wide siRNA screening identifies molecular targets mediating the sensitivity of pancreatic cancer cells to Aurora kinase inhibitors; Biochem Pharmacol 83:452-61, 2012, the teaching and contents of which are hereby incorporated by reference). P values were calculated using paired t test.

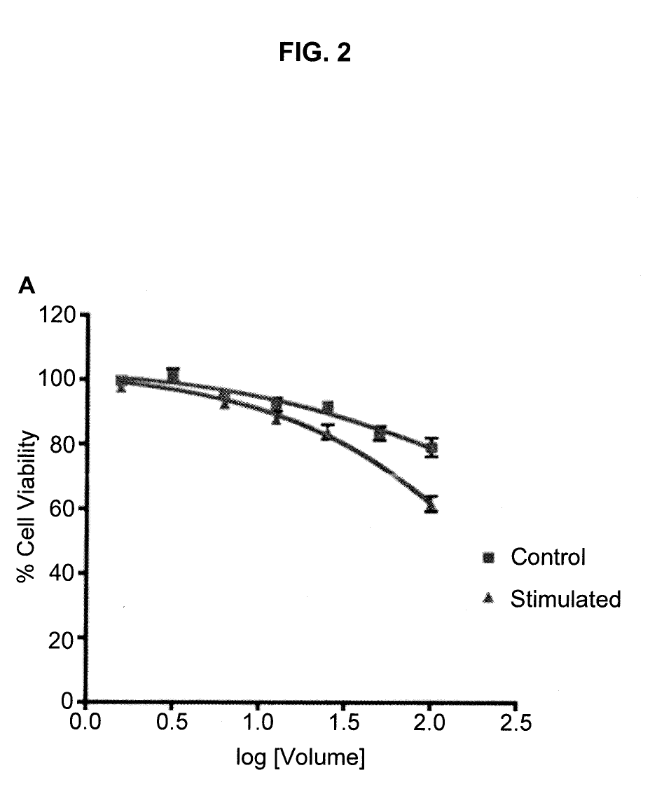

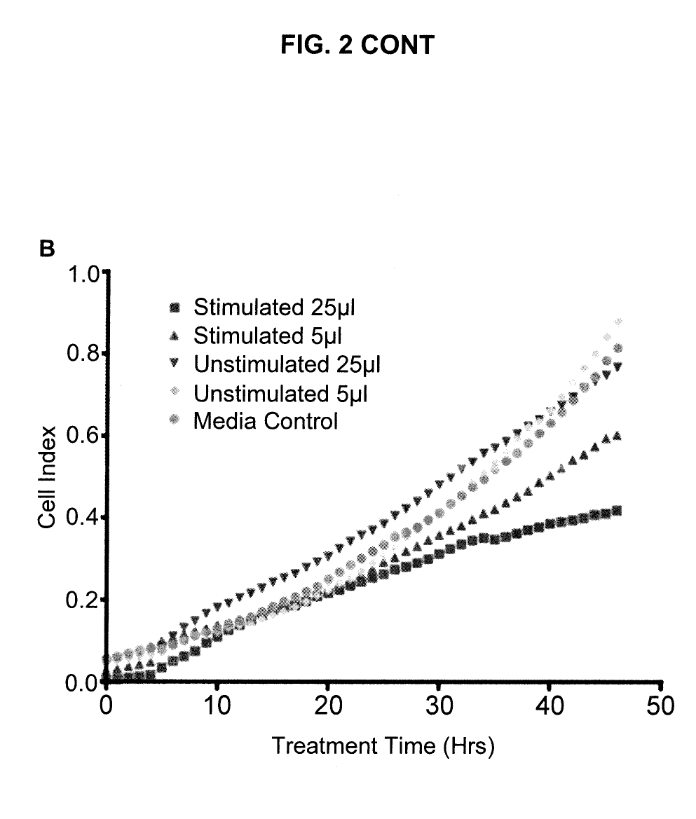

To evaluate the anti-tumor activity of the substances released from the stimulated muscles, the effect of the substances on the growth of two cancer cell lines, Mia PaCa-2 (a pancreatic cancer cell line) and MCF-7 (a breast cancer cell line) was measured. As shown in FIG. 2A, both stimulated and unstimulated substances showed inhibitory effect in the MCF-7 cells. However, the anti-proliferation activity of the substances obtained from the stimulated muscle was significantly higher than those obtained from the unstimulated muscles (P=0.02 for 100 uL volume using paired t test). Furthermore, at low concentrations, only the substance from stimulated muscles showed significant time-dependent inhibition of MCF-7 cell growth (FIG. 2B). Similar results were obtained for in Mia PaCa-2 cells (data not shown).

### Example 3

### Identification of the Substances Released from Stimulated Muscles

The F-substance was then identified using rat cytokine antibody arrays. The Rat Cytokine Array Panel A (Cat #ARY008) from R&D system (Minneapolis, Minn.) was used to probe cytokines in the substance isolated from stimulated and unstimulated rat muscles by following the procedures recommended by manufacturer. Briefly, the array membranes were first incubated in the block buffer for 1 hr. In the meantime, substances isolated from one stimulated or unstimulated muscle were mixed with the Detection Antibody Cocktail and incubated for 1 hour at room temperature. After removing the block buffer, the sample/antibody mixture were added to array membranes and incubated overnight at 4° C. After incubation, the membranes were washed 3 times with the Wash Buffer and then incubated the Steptavidin-HRP solution for 30 minutes at room temperature. The membranes were finally washed with Wash Buffer for 3 times and the bound antibodies were detected by chemiluminescence using the Immobilon™ Western Chemiluminescent HRP Substrate (Millipore, Billerica, Mass.). The membranes were quantified by densitometry. Statistical analysis was carried out by paired t test.

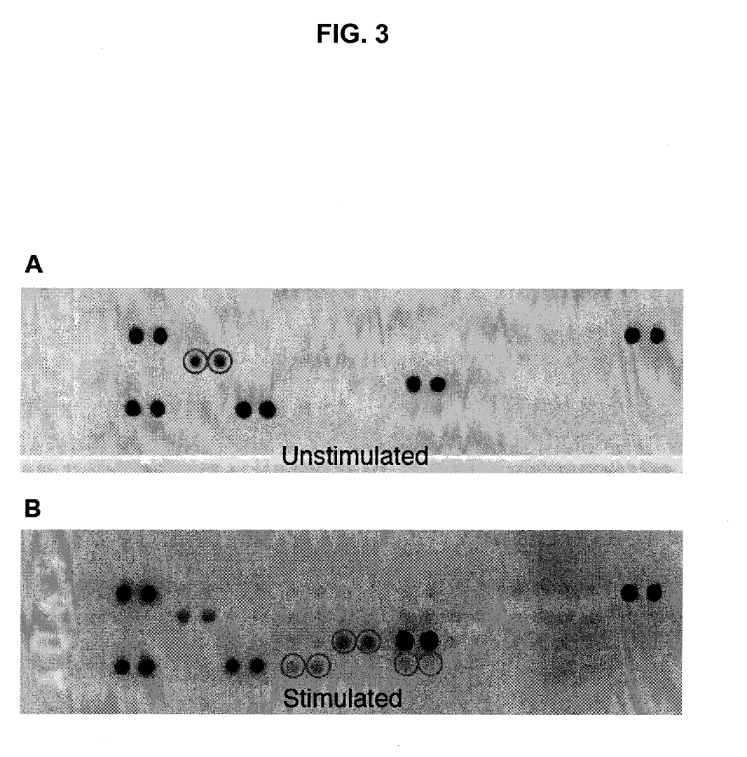

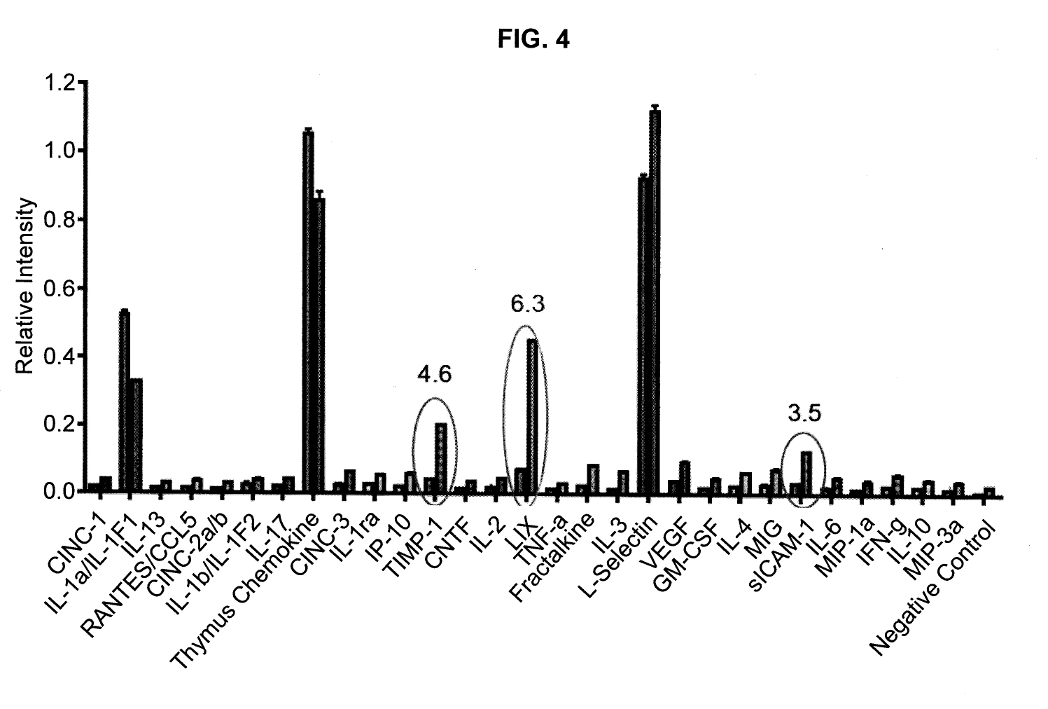

To identify the active molecule(s) that are responsible for the antitumor activity of the fatigue substance, an antibody array containing antibodies against 29 different rat cytokines was used to detect cytokines in the substance (FIG. 3). A total of 6 cytokines were detected in the samples isolated from unstimulated and stimulated muscles. Quantitation of the array membranes indicated that two of the 6 cytokines showed higher levels in the samples isolated from unstimulated muscles and 4 showed higher levels in the stimulated muscles, three (LIX, TIMP-1, and sICAM-1) of which showed greater than 3 fold difference (FIG. 4). LIX (also known as CXCL5) is 6-fold higher in the substances isolated from stimulated muscles than those from the unstimulated muscles. TIMP-1 is 4.6 fold higher and sICAM is 3.6 fold higher in the substances from the stimulated muscles. A paired t test showed that the differences in the levels of cytokines between the stimulated and unstimulated muscles were highly significant (P values equal 0.006, 0.007, and 0.033 for LIX, TIMP-1, and sICAM-1, respectively). These results indicated that cytokines released from contracting muscles might be responsible for the antitumor effect of F-Substance.

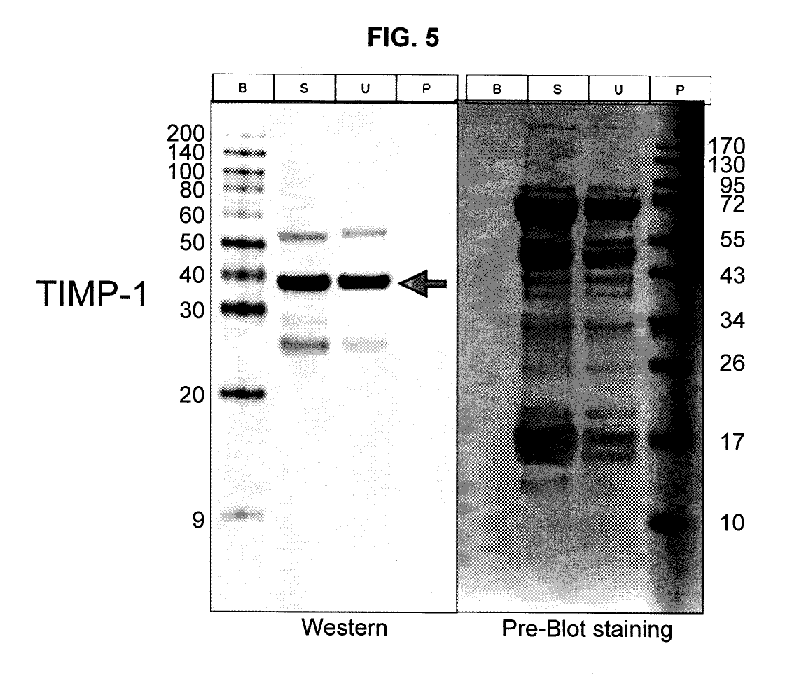

To further confirm the array results, a Western blotting analysis was prepared for each of the 3 cytokines that showed the most significant difference in the arrays. As shown in FIG. 5, the level of TIMP-1 is about 1.5 fold higher in the stimulated muscle extracts than in the unstimulated muscle.

We claim:

1. A composition for reducing tumor growth, comprising an isolated cytokine selected from the group consisting of LIX (or CXCL5), TIMP-1, sICAM, and combinations thereof.

2. The composition of claim 1, wherein the composition includes at least two isolated cytokines selected from the group consisting of LIX (or CXCL5), TIMP-1, and sICAM.

3. The composition of claim 1, wherein the isolated cytokine is lyophilized.

4. The composition of claim 3, wherein the lyophilized isolated cytokine is reconstituted in water, saline, PBS, plasma based solutions, or cell growth media.

5. A method of inhibiting tumor growth comprising the step of contacting a tumor with an isolated cytokine selected from the group consisting of LIX (or CXCL5), TIMP-1, sICAM, and combinations thereof.

6. The method of claim 5, wherein the tumor is contacted with at least two isolated cytokines selected from the group consisting of LIX (or CXCL5), TIMP-1, and sICAM.

7. The method of claim 5, wherein the isolated cytokine is lyophilized.

8. The method of claim 7, wherein the lyophilized isolated cytokine is reconstituted in water, saline, PBS, plasma based solutions, or cell growth media.

9. A method of inhibiting the proliferation of tumor cells comprising the step of contacting tumor cells with an isolated cytokine selected from the group consisting of LIX (or CXCL5), TIMP-1, sICAM, and combinations thereof.

10. The method of claim 9, wherein the tumor is contacted with at least two isolated cytokines selected from the group consisting of LIX (or CXCL5), TIMP-1, and sICAM.

11. The method of claim 9, wherein the isolated cytokine is lyophilized.

12. The method of claim 11, wherein the lyophilized isolated cytokine is reconstituted in water, saline, PBS, plasma based solutions, or cell growth media.
